(19) United States
(12) Patent Application Publication
(10) Pub. No.: US 2015/0327990 A1
(43) Pub. Date: Nov. 19, 2015

(54) OPTICAL AID AND RETINAL IMPLANT DEVICE THEREOF

(71) Applicant: National Chiao Tung University, Hsinchu City (TW)

(72) Inventors: Chung-Yu Wu, Hsinchu City (TW); Po-Kang Lin, Taipei City (TW); Chuan-Chin Chiao, Taipei City (TW); Jui-Wen Pan, Hsinchu City (TW)

(21) Appl. No.: 14/593,825

(22) Filed: Jan. 9, 2015

(30) Foreign Application Priority Data
May 16, 2014 (TW) 103117325

(51) Int. Cl. A61F 2/14

(52) U.S. Cl. CPC A61F 2/14

(57) ABSTRACT

An optical aid comprises a projecting device and a retinal implant device. The projecting device includes an image projector for outputting a projected image and a light generator for emitting an auxiliary light that differs in wavelength from the projected image. The retinal implant device includes an optical-to-electrical converter and an image sensor. The optical-to-electrical converter converts the auxiliary light received thereby into electricity for powering the image sensor. The image sensor is disposed in such a way that the auxiliary light is not received thereby, and generates an electrical stimulus signal associated with the projected image.

### CROSS-REFERENCE TO RELATED APPLICATIONS

This application claims priority of Taiwanese Application No. 103117325, filed on May 16, 2014, the contents of which are hereby incorporated by reference.

### BACKGROUND OF THE INVENTION

1. Field of the Invention

This invention relates to an optical device, and more particularly to an optical aid and a retinal implant device thereof.

2. Background Information

Retinitis Pigmentosa (RP) and age-related macular degeneration (AMD) are two diseases resulting from degeneration of photoreceptors of the retina. AMD is the main cause of adult blindness in western countries. No effective treatment is available for the blindness caused by these diseases. However, an artificial retina, which can replace the photoreceptors, has been employed to restore vision. Conventional artificial retina includes a solar cell for receiving an optical image and converting the same into current to stimulate the remaining ganglion cells, such that the vision of a patient blinded as a result of degeneration of photoreceptors can be restored. In the prior art, insufficient power supply of the solar cell is an issue, and use of wiring and routing for providing power raises safety concerns.

U.S. Patent Application Publication No. 2005/0090875 discloses a display and projector to be worn on the head of a patient for providing the eyeball with an image of sufficient intensity, so as to increase the output current from the solar cell. The proposed scheme, however, results in lower image contrast.

### SUMMARY OF THE INVENTION

Therefore, an object of the present invention is to provide an optical aid and a retinal implant device thereof that can overcome the aforesaid drawback associated with the prior art.

Accordingly, this invention provides an optical aid that includes a projecting device and a retinal implant device.

The projecting device includes an image projector and a light generator. The image projector is adapted to receive an external image, and outputs through optical projection a projected image associated with the external image. The light generator is operable to emit an auxiliary light that differs in wavelength from the projected image.

The retinal implant device includes an optical-to-electrical converter and an image sensor. The optical-to-electrical converter is to be disposed to receive the auxiliary light, and is capable of converting the auxiliary light into electricity. The image sensor is coupled to the optical-to-electrical converter to be powered by the electricity therefrom, is disposed to receive the projected image, and generates an electrical stimulus signal associated with the projected image.

The invention also provides the afore-described retinal implant device.

### BRIEF DESCRIPTION OF THE DRAWINGS

Other features and advantages of the present invention will become apparent in the following detailed description of the embodiment of this invention, with reference to the accompanying drawings, in which:

FIG. 1 is a block diagram illustrating the embodiment of an optical aid according to this invention;

FIG. 2 is a schematic diagram illustrating the embodiment; and

FIG. 3 is an exploded schematic diagram illustrating a retinal implant device of the embodiment.

### DETAILED DESCRIPTION

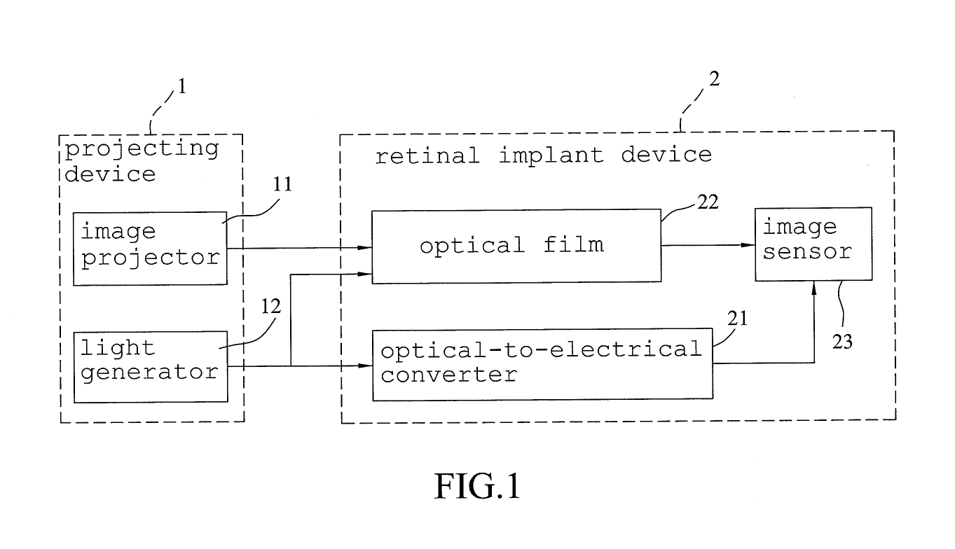

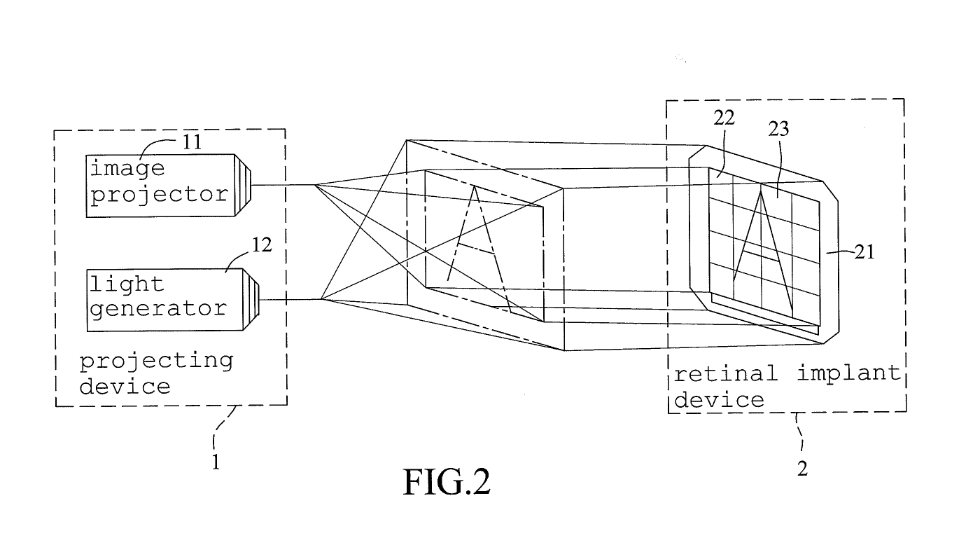

Referring to FIGS. 1 to 3, the embodiment of an optical aid according to this invention includes a projecting device 1 and a retinal implant device 2.

The projecting device 1 includes an image projector 11 and a light generator 12.

The image projector 11 is adapted for receiving an external image, and outputs through optical projection a projected image associated with the external image.

The light generator 12 is operable to emit an auxiliary light. In this embodiment, the auxiliary light has an optical characteristic different from that of the projected image. For example, the auxiliary light is infrared light, and the projected image is composed of light rays within a wavelength range different from that of the infrared light. The auxiliary light serves as a power source of the retinal implant device 2 in this invention, as will be apparent in the following description.

The retinal implant device 2 in this embodiment is an implantable biomedical chip to be disposed through surgery in a sub-retina region of a patient's eyeball. The retinal implant device 2 includes an optical-to-electrical converter 21 and an image sensor 23.

The optical-to-electrical converter 21 is disposed to receive the auxiliary light, and is capable of converting the auxiliary light into electricity. In this embodiment, the optical-to-electrical converter 21 is a solar cell. The image sensor 23 is coupled to the optical-to-electrical converter 21 to be powered by the electricity therefrom, and is disposed for generating an electrical stimulus signal associated with the projected image. The electrical stimulus signal is for restoring the patient's vision.

In this embodiment, the retinal implant device 2 further includes an optical film 22 to be disposed between the projecting device 1 and the optical sensor 23. The optical film 22 is capable of preventing passage therethrough of any auxiliary light incident thereupon, and permitting passage of the projected image therethrough. In other words, the image sensor 23, which is disposed behind the optical film 22, receives the projected image that passes through the optical film 22 in order to generate the electrical stimulus signal based on the projected image. Ideally speaking, the image sensor 23 and the optical film 22 are so sized that the image sensor 23 receives the complete projected image via the optical film 22 without receiving any auxiliary light. Since the auxiliary light does not reach the image sensor 23, generation of additional photocurrent and thus increase of power consumption and reduction of image contrast can be prevented.

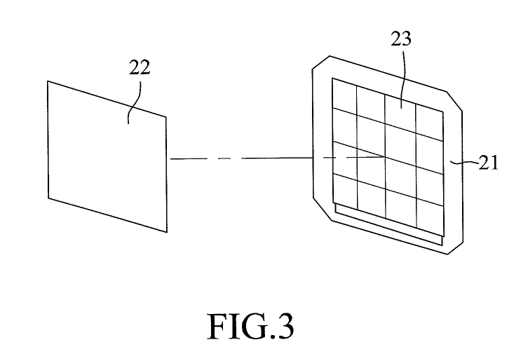

Herein, an illumination range of the projected image and an illumination range of the auxiliary light overlap at least in part, and the optical film 22 is to span the illumination range of the projected image so as to prevent passage of any auxiliary light to the optical sensor 23. In this embodiment, the illumination range of the auxiliary light is greater than and covers the entire illumination range of the projected image, i.e., the projected image is completely overlapped by a portion of the auxiliary light, while at least one other portion of the auxiliary light can directly illuminate upon the optical-to-electrical converter 21. In one implementation, as shown in FIG. 3, the optical-to-electrical converter 21 surrounds the image sensor 23 (i.e., the optical-to-electrical converter 21 and the image sensor 23 occupy different regions of the retinal implant device 2), and is substantially not covered by the optical film 22 so as to directly receive the auxiliary light and attain high conversion efficiency.

In view of the above, the embodiment of this invention uses the auxiliary light that is different in wavelength from the projected image to supply power to the retinal implant device 2 in a way that input of the optical sensor 23 is substantially unaffected by the presence of the auxiliary light so as to maintain image contrast while providing sufficient power supply to the retinal implant device 2. The embodiment of this invention further uses the optical film 22 as an additional measure to prevent the auxiliary light from interfering with the input to the optical sensor 23.

It should be noted herein that in some instances of this invention, the auxiliary light may emit in a manner that it does not overlap with the project image and only illuminates the optical-to-electrical converter 21, in which case the optical film 22 becomes non-essential. In addition, in other instances of this invention, the image sensor 23 may be able to have a low sensitivity to waves falling within the wavelength range of the auxiliary light, such that even if the auxiliary light overlaps with the projected image, and even if the optical film 22 is not present, the merits of this invention may still be achieved.

While the present invention has been described in connection with what is considered the most practical embodiment, it is understood that this invention is not limited to the disclosed embodiment but is intended to cover various arrangements included within the spirit and scope of the broadest interpretation and equivalent arrangements.

1. An optical aid comprising:
a projecting device including
an image projector adapted for receiving an external image, and outputting through optical projection a projected image that is associated with the external image, and
a light generator operable to emit an auxiliary light that is different in wavelength from the projected image; and
a retinal implant device including
an optical-to-electrical converter to be disposed to receive the auxiliary light, and capable of converting the auxiliary light into electricity, and
an image sensor coupled to said optical-to-electrical converter to be powered by the electricity therefrom, disposed to receive the projected image, and generating an electrical stimulus signal associated with the projected image.

2. The optical aid of claim 1, further comprising an optical film to be disposed between said projecting device and said image sensor, and being capable of preventing passage therethrough of any auxiliary light incident thereupon, and permitting passage of the projected image therethrough.

3. The optical aid of claim 2, wherein an illumination range of the projected image and an illumination range of the auxiliary light overlap at least in part, and said optical film is to span the illumination range of the projected image.

4. The optical aid of claim 3, wherein the illumination range of the auxiliary light is greater than and covers the illumination range of the projected image.

5. The optical aid of claim 2, wherein said optical-to-electrical converter is substantially not covered by said optical film so as to receive the auxiliary light.

6. The optical aid of claim 1, wherein said image sensor is surrounded by said optical-to-electrical converter.

7. The optical aid of claim 1, wherein the auxiliary light is infrared light, and a wavelength range of the projected image is different from that of the infrared light.

8. The optical aid of claim 1, wherein said optical-to-electrical converter is a solar cell.

9. A retinal implant device for receiving an auxiliary light and a projected image that differ from each other in wavelength, said retinal implant device comprising:
an optical-to-electrical converter to be disposed to receive the auxiliary light, and capable of converting the auxiliary light into electricity, and
an image sensor coupled to said optical-to-electrical converter to be powered by the electricity therefrom, disposed to receive the projected image, and generating an electrical stimulus signal associated with the projected image.

10. The retinal implant device of claim 9, further comprising an optical film disposed in front of said image sensor, and being capable of preventing passage therethrough of any auxiliary light incident thereupon, and permitting passage of the projected image therethrough.

11. The retinal implant device of claim 10, wherein said optical film is to span an illumination range of the projected image, which overlaps at least in part with an illumination range of the auxiliary light.

12. The optical aid of claim 10, wherein said optical-to-electrical converter is substantially not covered by said optical film so as to receive the auxiliary light.

13. The retinal implant device of claim 9, wherein said image sensor is surrounded by said optical-to-electrical converter.

14. The retinal implant device of claim 9, wherein said optical-to-electrical converter is a solar cell.
